(19) United States
(12) Patent Application Publication
(10) Pub. No.: US 2020/0253959 A1
(43) Pub. Date: Aug. 13, 2020

(54) COMPOUNDS AND PHARMACEUTICAL COMPOSITIONS THEREOF FOR USE IN THE TREATMENT OF FIBROTIC DISEASES

(71) Applicant: Galapagos NV, Mechelen (BE)

(72) Inventors: Elisabeth Martina VAN DER AAR, Mechelen (BE); Olivier Frans Jozef Maria VAN DE STEEN, Laarne (BE)

(21) Appl. No.: 16/637,590

(22) PCT Filed: Aug. 8, 2018
(86) PCT No.: PCT/EP2018/071487

§ 371 Date: Feb. 7, 2020

(30) Foreign Application Priority Data
Aug. 9, 2017 (GB) 1712742.4
May 2, 2018 (GB) 1807222.3

(51) Int. Cl. A61K 31/496; A61P 11/00

(52) U.S. Cl. CPC A61K 31/496; A61B 6/032; A61P 11/00

(57) ABSTRACT

The present invention relates to the use of the compound according to Formula I in the prophylaxis and/or treatment of fibrotic diseases, more particularly idiopathic pulmonary fibrosis.

### FIELD OF THE INVENTION

The present invention relates to the use of the compound according to Formula I in the treatment of fibrotic diseases, more particularly idiopathic pulmonary fibrosis. In particular, the compound according to Formula I inhibits autotaxin (ATX), also known as ectonucleotide pyrophosphatase/phosphodiesterase 2 (NPP2 or ENPP2), that is involved in fibrotic diseases, proliferative diseases, inflammatory diseases, autoimmune diseases, respiratory diseases, cardiovascular diseases, neurodegenerative diseases, dermatological disorders, and/or abnormal angiogenesis associated diseases.

### BACKGROUND OF THE INVENTION

Autotaxin is the main source of lysophosphatidic acid (LPA) in blood, and blood LPA and ATX levels have been shown to be strongly correlated in humans. In turn LPA is associated to a range of diseases including fibrotic diseases, proliferative diseases, inflammatory diseases, autoimmune diseases, respiratory diseases, cardiovascular diseases, neurodegenerative diseases, dermatological disorders, and/or abnormal angiogenesis associated diseases, and more particularly IPF.

Therefore inhibiting ATX may be useful in the treatment of these diseases, in particular IPF, and ATX inhibitors have been described in WO 2014/139882 and WO 2014/202458.

Idiopathic pulmonary fibrosis (IPF) is a chronic, progressive, severely debilitating, and ultimately lethal lung disease predominantly affecting elderly male smokers or ex-smokers with a median age of 65-70 years (Cordier & Cottin, 2013). The disease is characterized by progressive worsening of dyspnea and lung function and is associated with a poor prognosis (i.e., median survival of 2-5 years following diagnosis) (Meltzer and Noble, 2008; Raghu et al., 2011; Ley et al., 2011).

The estimated IPF prevalence ranges from 14.0 to 27.9 cases per 100,000 population in the United States (US) and from 1.3 to 23.4 cases per 100,000 population in Europe (data from 1990-2011). The estimated IPF incidence in these studies ranges from 6.8-8.8 cases per 100,000 population in the US and from 0.2-7.4 cases per 100,000 population in Europe (Perez et al., 2010; Raghu et al., 2006; Thomeer et al., 2001).

Over the past decade, extensive research has been conducted to address the unmet medical need for effective IPF treatment. Two treatments, pirfenidone and nintedanib, targeting the biologic processes that drive fibrosis are currently approved in the European Union (EU) and US.

Pirfenidone, marketed as Esbriet® was approved in the EU in 2011 and in the US in 2014. It was the first drug to be licensed specifically for IPF, and was shown to improve progression-free survival and to slow the decline in forced vital capacity (FVC). (King et al., 2014; Noble et al., 2011).

Nintedanib, marketed as Ofev® was approved in the US in 2014 and in the EU in 2015. It was initially developed as an anticancer agent, and was shown to significantly reduce the decline in FVC compared with placebo, with also a trend towards a reduced death rate although the studies were not powered to detect differences in mortality (Richeldi et al., 2014).

Both treatments appear to slow disease progression, but they do not stop said progression and are frequently associated with side effects potentially limiting the use in clinical practice (Raghu et al., 2015). Consequently, in order to avoid overdosing which may result in undesirable side effects, or underdosing which may compromise the treatment efficacy, it is essential to be able to measure the patient response and thus identify the optimal dose.

WO 2014/125059 discloses a general method for determining a respiratory condition based on functional respiratory imaging, however without any fibrotic disease specific treatment agent, more particularly IPF.

There thus remains a significant unmet medical need for the investigation and development of novel IPF treatments, more particularly treatments addressing the specific patient profile and requirements.

### SUMMARY OF THE INVENTION

The present invention relates to the use of the compound according to Formula I in the treatment of fibrotic diseases, more particularly idiopathic pulmonary fibrosis.

In a first aspect of the invention is provided a method of treatment of fibrotic diseases, which method comprises the steps of:
a) obtaining three-dimensional image data of a subject's respiratory system, which image data has been acquired during an assessment period, wherein the assessment period comprises a breathing cycle and the image data comprise high-resolution computer tomography (CT) images at functional residual capacity (FRC) and high-resolution computer tomography (CT) images at total lung capacity (TLC);
b) calculating a specific three-dimensional structural model of the subject's respiratory system using the three-dimensional image data of step a) to determine one or more outcome parameters as described in WO 2014/125059, the content of which are herein incorporated by reference;
c) administering a daily dose of between 100 mg to 1000 mg to the subject of one or more compounds disclosed in WO 2014/139882 and WO 2014/202458;
d) repeating steps a and b above after administration of the compounds;
e) comparing the three-dimensional structural models and/or outcome parameters of step b) and d) with each other;
f) calculating the response to the treatment of said subject from step e);
g) determining whether the dose of the compound according to Formula I should be increased, decreased or maintained at the same level based on the results obtained in step f).

In a second aspect of the invention is provided a method of treatment of fibrotic diseases, which method comprises the steps of:
a) obtaining three-dimensional image data of a subject's respiratory system, which image data has been acquired during an assessment period, wherein the assessment period comprises a breathing cycle and the image data comprise high-resolution computer tomography (CT) images at functional residual capacity (FRC) and high-resolution computer tomography (CT) images at total lung capacity (TLC);
b) calculating a specific three-dimensional structural model of the subject's respiratory system using the three-dimensional image data of step a) to determine one or more outcome parameters as described in WO 2014/125059, the content of which are herein incorporated by reference;
c) administering a daily dose of between 100 mg to 1000 mg to the subject of a compound according to Formula I (Cpd 1), or a pharmaceutically acceptable salt thereof:

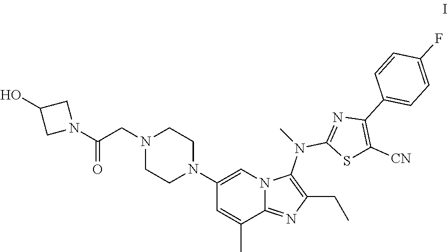 [SMILES: CCc1nc2c(C)cc(N3CCN(CC(=O)N4CC(O)C4)CC3)cn2c1N(C)c1nc(-c2ccc(F)cc2)c(C#N)s1]

d) repeating steps a and b above after administration of the compound;
e) comparing the three-dimensional structural models and/or outcome parameters of step b) and d) with each other;
f) calculating the response to the treatment of said subject from step e);
g) determining whether the dose of the compound according to Formula I should be increased, decreased or maintained at the same level based on the results obtained in step f).

In a more particular aspect the fibrotic disease is IPF.

In a further aspect, the present invention provides pharmaceutical compositions comprising the compound of the invention, and a pharmaceutical carrier, excipient or diluent for use in the treatment of fibrotic diseases. In a particular aspect, the pharmaceutical composition may additionally comprise further therapeutically active ingredients suitable for use in combination with the compounds of the invention. In a more particular aspect, the further therapeutically active ingredient is an agent for the treatment of fibrotic disease. In a most particular embodiment, the fibrotic disease is IPF.

Moreover, the compounds of the invention, useful in the pharmaceutical compositions and treatment methods disclosed herein, are pharmaceutically acceptable as prepared and used.

In a further aspect of the invention, this invention provides a method of treating a mammal, in particular humans, afflicted with a condition selected from among those listed herein, particularly fibrotic diseases, and more particularly IPF, which method comprises administering an effective amount of the pharmaceutical composition or compounds of the invention as described herein.

The present invention also provides pharmaceutical compositions comprising a compound of the invention, and a suitable pharmaceutical carrier, excipient or diluent for use in the treatment of fibrotic diseases, more particularly IPF.

Other objects and advantages will become apparent to those skilled in the art from a consideration of the ensuing detailed description.

It will be appreciated that compounds of the invention may be metabolized to yield biologically active metabolites.

### BRIEF DESCRIPTION OF THE DRAWINGS

FIG. 1: shows the variation in serum LPA 18:2 levels for the compound according to Formula I (Cpd 1) dosed at 600 mg qd (filled squares) vs the placebo (filled diamonds) over the 12 weeks study and follow up visit.

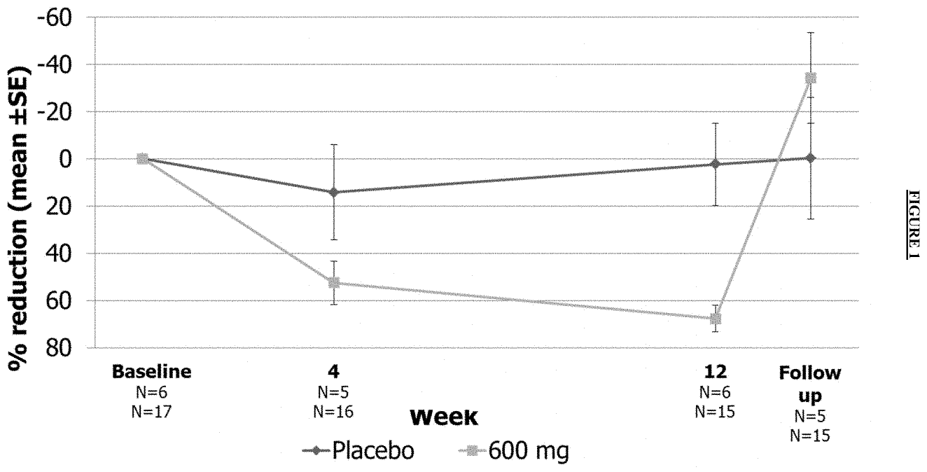

FIG. 2: shows the variation in Forced vital capacity (FVC) for the compound according to Formula I (Cpd 1) dosed at 600 mg qd (filled circles) vs the placebo (filled triangle) over the 12 weeks study and follow up visit.

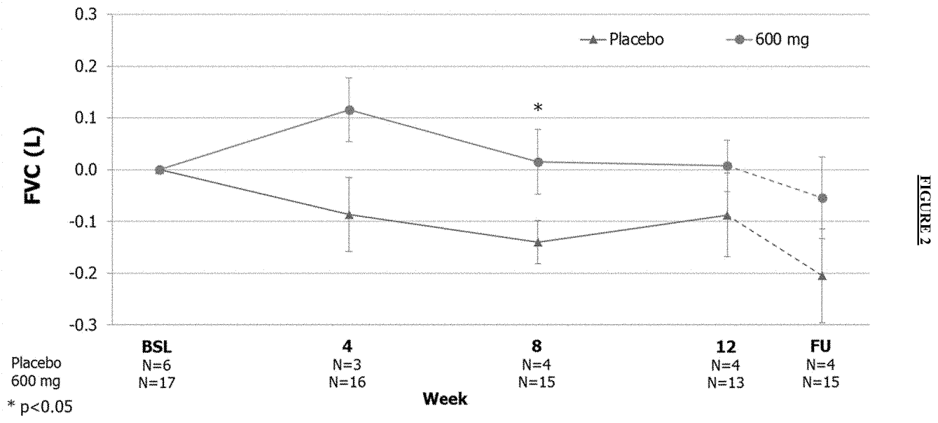

FIG. 3: shows the comparison in specific airway volume between the placebo and the compound according to Formula I (Cpd 1) dosed at 600 mg qd at baseline and at 12 weeks.

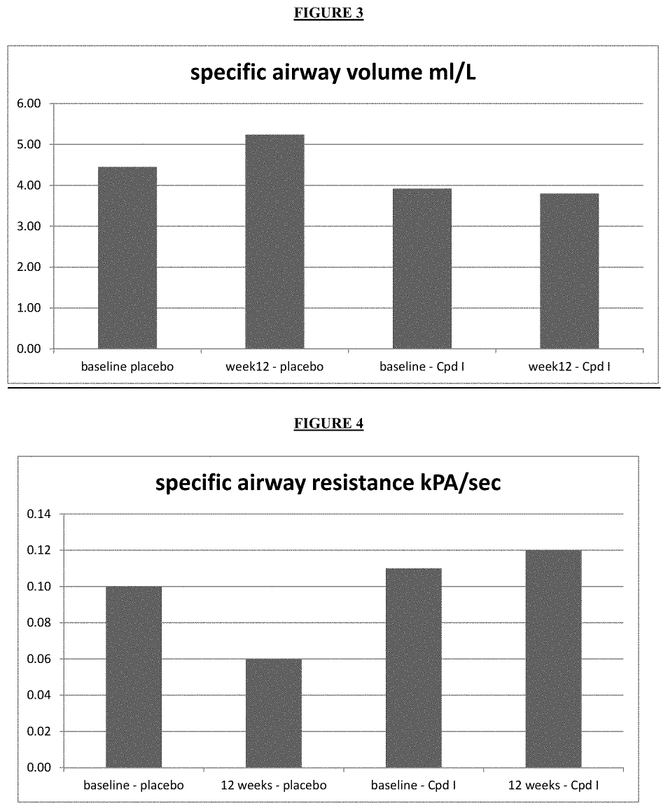

FIG. 4: shows the comparison in specific airway resistance between the placebo and the compound according to Formula I (Cpd 1) dosed at 600 mg qd at baseline and at 12 weeks.

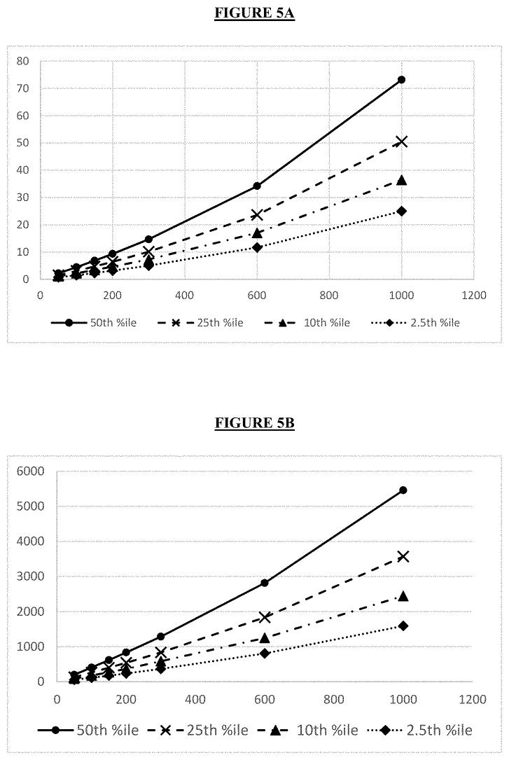

FIG. 5: shows percentiles (solid line: 50^{th}% ile; regular dashed line: 25^{th}% ile; irregular dashed line: 10^{th}% ile; dotted line: 2.5^{th}% ile) of individual simulated steady state area under the curve (5A), maximum plasma concentration (5B), area under the effect curve (5C), and maximal plasma lysophosphatidic acid 18:2 reduction (5D) at doses of compound according to Formula I between 50 and 1000 mg daily as calculated for example 2.

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles ‘a’ and ‘an’ may be used herein to refer to one or to more than one (i.e. at least one) of the grammatical objects of the article. By way of example ‘an analogue’ means one analogue or more than one analogue.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Prodrugs’ refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding. Conventional solvents include water, EtOH, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans The terms ‘human’, ‘patient’ and ‘subject’ are used interchangeably herein.

‘Effective amount’ means the amount of a compound of the invention that, when administered to a subject for treating a disease, is sufficient to effect such treatment for the disease. The “effective amount” can vary depending on the compound, the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein the term ‘fibrotic diseases’ refers to diseases characterized by excessive scarring due to excessive production, deposition, and contraction of extracellular matrix, and are that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract. In particular, the term fibrotic diseases refers to idiopathic pulmonary fibrosis (IPF); cystic fibrosis, other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease); radiation induced fibrosis; chronic obstructive pulmonary disease (COPD); scleroderma; bleomycin induced pulmonary fibrosis; chronic asthma; silicosis; asbestos induced pulmonary fibrosis; acute respiratory distress syndrome (ARDS); kidney fibrosis; tubulointerstitium fibrosis; glomerular nephritis; focal segmental glomerular sclerosis; IgA nephropathy; hypertension; Alport; gut fibrosis; liver fibrosis; cirrhosis; alcohol induced liver fibrosis; toxic/drug induced liver fibrosis; hemochromatosis; nonalcoholic steatohepatitis (NASH); biliary duct injury; primary biliary cirrhosis; infection induced liver fibrosis; viral induced liver fibrosis; and autoimmune hepatitis; corneal scarring; hypertrophic scarring; Dupuytren disease, keloids, cutaneous fibrosis; cutaneous scleroderma; systemic sclerosis, spinal cord injury/fibrosis; myelofibrosis; vascular restenosis; atherosclerosis; arteriosclerosis; Wegener's granulomatosis; Peyronie's disease, or chronic lymphocytic. More particularly, the term “fibrotic diseases” refers to idiopathic pulmonary fibrosis (IPF).

‘Compound(s) of the invention’, and equivalent expressions, are meant to embrace compounds of the Formula(e) as herein described, which expression includes the pharmaceutically acceptable salts, and the solvates, e.g. hydrates, and the solvates of the pharmaceutically acceptable salts where the context so permits Similarly, reference to intermediates, whether or not they themselves are claimed, is meant to embrace their salts, and solvates, where the context so permits.

When ranges are referred to herein, for example but without limitation, C₁-₈ alkyl, the citation of a range should be considered a representation of each member of said range.

Other derivatives of the compounds of this invention have activity in both their acid and acid derivative forms, but in the acid sensitive form often offers advantages of solubility, tissue compatibility, or delayed release in the mammalian organism (Bundgard, H, 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particularly useful prodrugs. In some cases it is desirable to prepare double ester type prodrugs such as (acyloxy)alkyl esters or ((alkoxycarbonyl)oxy)alkylesters. Particular such prodrugs are the C₁-₈ alkyl, C₂-₈ alkenyl, C₆-₁₀ optionally substituted aryl, and (C₆-₁₀ aryl)-(C₁₋₄ alkyl) esters of the compounds of the invention.

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitro (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e. as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

The compounds of the invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

It will be appreciated that compounds of the invention may be metabolized to yield biologically active metabolites.

### THE INVENTION

The present invention relates to the use of the compound according to Formula I in the treatment of fibrotic diseases, more particularly idiopathic pulmonary fibrosis.

In a first aspect of the invention is provided a method of treatment of fibrotic diseases, which method comprises the steps of:
a) obtaining three-dimensional image data of a subject's respiratory system, which image data has been acquired during an assessment period, wherein the assessment period comprises a breathing cycle and the image data comprise high-resolution computer tomography (CT) images at functional residual capacity (FRC) and high-resolution computer tomography (CT) images at total lung capacity (TLC);
b) calculating a specific three-dimensional structural model of the subject's respiratory system using the three-dimensional image data of step a) to determine one or more outcome parameters as described in WO 2014/125059, the content of which are herein incorporated by reference;
c) administering a daily dose of between 100 mg to 1000 mg to the subject of one or more compounds disclosed in WO 2014/139882 and WO 2014/202458;
d) repeating steps a and b above after administration of the compounds;
e) comparing the three-dimensional structural models and/or outcome parameters of step b) and d) with each other;
f) calculating the response to the treatment of said subject from step e);
g) determining whether the dose of the compound according to Formula I should be increased, decreased or maintained at the same level based on the results obtained in step f).

In a second aspect of the invention is provided a method of treatment of fibrotic diseases, which method comprises the steps of:
a) obtaining three-dimensional image data of a subject's respiratory system, which image data has been acquired during an assessment period, wherein the assessment period comprises a breathing cycle and the image data comprise high-resolution computer tomography (CT) images at functional residual capacity (FRC) and high-resolution computer tomography (CT) images at total lung capacity (TLC);
b) calculating a specific three-dimensional structural model of the subject's respiratory system using the three-dimensional image data of step a) to determine one or more outcome parameters as described in WO 2014/125059, the content of which are herein incorporated by reference;
c) administering a daily dose of between 100 mg to 1000 mg to the subject of a compound according to Formula I (Cpd 1), or a pharmaceutically acceptable salt thereof:

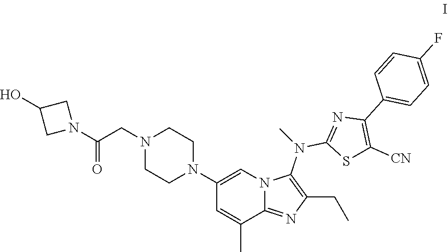 [SMILES: CCc1nc2c(C)cc(N3CCN(CC(=O)N4CC(O)C4)CC3)cn2c1N(C)c1nc(-c2ccc(F)cc2)c(C#N)s1]

d) repeating steps a and b above after administration of the compound;
e) comparing the three-dimensional structural models and/or outcome parameters of step b) and d) with each other;
f) calculating the response to the treatment of said subject from step e);
g) determining whether the dose of the compound according to Formula I should be increased, decreased or maintained at the same level based on the results obtained in step f).

In one embodiment, step d above is performed after 4 weeks, after 8 weeks, or after 12 weeks administration of the compound according to formula I. In a more particular embodiment, step d is performed after 12 weeks administration of the compound according to Formula I.

In one embodiment, the specific three-dimensional structural model of the subject's respiratory system comprises a three-dimensional structural model of the subject's lung lobar structure and a three-dimensional structural model of the subject's airway structure.

In one embodiment, the one or more outcome parameters comprise the lobar volume, preferably at FRC and TLC; or wherein the one or more outcome parameters comprise the airway volume, preferably at FRC and TLC; or wherein the one or more outcome parameters comprise lobar emphysema; or wherein the one or more outcome parameters comprise lobar blood vessel volume; or wherein the one or more outcome parameters comprise the airway wall thickness; or wherein the one or more outcome parameters comprise the airway resistance, preferably at FRC and TLC; or wherein the one or more outcome parameters comprise the airway volume and/or resistance. In a particular embodiment, the one or more outcome parameters comprise the airway volume and/or resistance.

In one embodiment, the compound is administered over a period of at least 1 week, at least 2 weeks, at least 4 weeks, at least 8 weeks or at least 12 weeks. In a particular embodiment, the compound of the invention is administered over a period of at least 12 weeks.

In one embodiment, the daily dose of the compound is 150, 300, 600, or 750 mg per day. In a particular embodiment, the compound is administered once a day (q.d.). In another particular embodiment, the compound is administered twice a day (b.i.d.). In a more particular embodiment, the compound of the invention is administered once a day at a dose of 600 mg. In a most particular embodiment, compound of the invention is administered once a day at a dose of 600 mg in the morning. Alternatively, in a more particular embodiment, the compound of the invention is administered once daily at a dose of 200 mg, most particularly in the morning.

In one embodiment, the method comprises measuring the forced vital capacity FVC in the subject, wherein the FVC does not decrease following treatment. In a particular embodiment, FVC does not decrease over a period of 12 weeks of treatment.

In another embodiment, the method comprises measuring the forced vital capacity FVC in the subject, wherein the FVC increases by at least 1mL, at least 2 mL, at least 3 mL, at least 4 mL, at least 5 mL, at least 6 mL, at least 7 mL or at least 8 mL. In a particular embodiment, the FVC increases by at least 1mL, at least 2 mL, at least 3 mL, at least 4 mL, at least 5 mL, at least 6 mL, at least 7 mL or at least 8 mL over a period of 12 weeks.

In one embodiment, the method comprises measuring the airway volume wherein said airway volume decrease is no more than 5 mL/L, no more than 4 mL/l, or no more than 3 mL/L. In a particular embodiment the airway volume decrease is no more than 5 mL/L, no more than 4 mL/l, or no more than 3 mL/L after 12 weeks.

In one embodiment, the method comprises measuring the airway volume wherein said airway resistance increase is at least 0.05 kPa/s, at least 0.06 kPa/s, at least 0.07 kPa/s, at least 0.08 kPa/s, at least 0.09kPa/s, or at least 1.0 kPa/s. In a particular embodiment the airway volume increase is at least 0.05 kPa/s, at least 0.06 kPa/s, at least 0.07 kPa/s, at least 0.08 kPa/s, at least 0.09kPa/s, or at least 1.0 kPa/s after 12 weeks.

In a more particular aspect the fibrotic disease is IPF.

In one embodiment a compound of the invention is not an isotopic variant.

In one aspect a compound of the invention according to any one of the embodiments herein described is present as the free base.

In one aspect a compound of the invention according to any one of the embodiments herein described is a pharmaceutically acceptable salt.

In one aspect a compound of the invention according to any one of the embodiments herein described is a solvate of the compound.

In one aspect a compound of the invention according to any one of the embodiments herein described is a solvate of a pharmaceutically acceptable salt of a compound.

While specified groups for each embodiment have generally been listed above separately, a compound of the invention includes one in which several or each embodiment in the above Formula, as well as other formulae presented herein, is selected from one or more of particular members or groups designated respectively, for each variable. Therefore, this invention is intended to include all combinations of such embodiments within its scope.

While specified groups for each embodiment have generally been listed above separately, a compound of the invention may be one for which one or more variables (for example, R groups) is selected from one or more embodiments according to any of the Formula(e) listed above. Therefore, the present invention is intended to include all combinations of variables from any of the disclosed embodiments within its scope.

Alternatively, the exclusion of one or more of the specified variables from a group or an embodiment, or combinations thereof is also contemplated by the present invention.

In certain aspects, the present invention provides prodrugs and derivatives of the compounds according to the formulae above. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (Bundgard, H, 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Particularly useful are the C₁ to C₈ alkyl, C₂-C₈ alkenyl, aryl, C₇-C₁₂ substituted aryl, and C₇-C₁₂ arylalkyl esters of the compounds of the invention.

### Pharmaceutical Compositions

When employed as a pharmaceutical, a compound of the invention is typically administered in the form of a pharmaceutical composition. Such compositions can be prepared in a manner well known in the pharmaceutical art and comprise at least one active compound of the invention according to Formula I. Generally, a compound of the invention is administered in a pharmaceutically effective amount. The amount of compound of the invention actually administered will typically be determined by a physician, in the light of the relevant circumstances, including the condition to be treated, the chosen route of administration, the actual compound of the invention administered, the age, weight, and response of the individual patient, the severity of the patient's symptoms, and the like.

The pharmaceutical compositions of this invention can be administered by a variety of routes including oral, rectal, transdermal, subcutaneous, intra-articular, intravenous, intramuscular, and intranasal. Depending on the intended route of delivery, a compound of the invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the compound of the invention according to Formula I is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compound of the inventions of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint or orange flavoring.

Injectable compositions are typically based upon injectable sterile saline or phosphate-buffered saline or other injectable carriers known in the art. As before, the active compound of the invention according to Formula I in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

A compound of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pa., which is incorporated herein by reference.

A compound of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

A compound of the invention according to Formula I may be admixed as a dry powder with a dry gelatin binder in an approximate 1:2 weight ratio. A minor amount of magnesium stearate may be added as a lubricant. The mixture may be formed into 240-270 mg tablets (80-90 mg of active compound of the invention according to Formula I per tablet) in a tablet press.

### Formulation 2—Capsules

A compound of the invention according to Formula I may be admixed as a dry powder with a starch diluent in an approximate 1:1 weight ratio. The mixture may be filled into 250 mg capsules (125 mg of active compound of the invention according to Formula I per capsule).

### Formulation 3—Liquid

A compound of the invention according to Formula I (125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring. Further sufficient water may be then added to produce a total volume of 5 mL.

### Formulation 4—Tablets

A compound of the invention according to Formula I may be admixed as a dry powder with a dry gelatin binder in an approximate 1:2 weight ratio. A minor amount of magnesium stearate may be added as a lubricant. The mixture may be formed into 450-900 mg tablets (150-300 mg of active compound of the invention according to Formula I) in a tablet press.

### Formulation 5—Injection

A compound of the invention according to Formula I may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of A compound of the invention according to Formula I (50 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### Methods of Treatment

In one embodiment, the present invention provides compounds of the invention, or pharmaceutical compositions comprising a compound of the invention for use the treatment of fibrotic diseases. In a particular embodiment the fibrotic disease is IPF.

In another embodiment, the present invention provides compounds of the invention, or pharmaceutical compositions comprising a compound of the invention for use in the manufacture of a medicament for use in treatment of fibrotic diseases. In a particular embodiment the fibrotic disease is IPF.

In additional method of treatment aspects, this invention provides methods of treatment of a mammal afflicted with fibrotic diseases, which methods comprise the administration of an effective amount of a compound of the invention or one or more of the pharmaceutical compositions herein described for the treatment of said condition. In a particular embodiment the fibrotic disease is IPF.

In one embodiment, the present invention provides pharmaceutical compositions comprising a compound of the invention, and another therapeutic agent. In a particular embodiment, the other therapeutic agent is a fibrotic diseases treatment agent. In a particular embodiment the fibrotic disease is IPF.

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 1 g/day for a 40 to 80 kg human patient.

For the treatment of long-term conditions, such as degenerative conditions, the regimen for treatment usually stretches over many months or years so oral dosing is preferred for patient convenience and tolerance. With oral dosing, one to four (1-4) regular doses daily, especially one to three (1-3) regular doses daily, typically one to two (1-2) regular doses daily, and most typically one (1) regular dose daily are representative regimens. More in particular, the compound is administered once daily in the morning. Alternatively for long lasting effect drugs, with oral dosing, once every other week, once weekly, and once a day are representative regimens. In particular, dosage regimen can be every 1-14 days, more particularly 1-10 days, even more particularly 1-7 days, and most particularly 1-3 days.

Using these dosing patterns, each dose provides from about 1 to about 1000 mg of a compound of the invention, with particular doses each providing from about 10 to about 500 mg and especially about 30 to about 250 mg. In one embodiment, the daily oral dose of a compound of the invention and in particular of a compound according to Formula I is from 100 mg to 700 mg or from 200 mg to 600 mg, such as 200 mg, 300 mg, 400 mg, 500 mg or 600 mg. In a particular embodiment, the daily oral dose of a compound according to Formula I is 200, 400 or 600 mg.

In a specific embodiment, the compound according to Formula I is administered in a once daily dose of 200 mg. In an alternative specific embodiment, the compound according to Formula I is administered in a once daily dose of 600 mg. In a more specific embodiment, said once daily dose is administered orally.

In another embodiment, dosage is lowered to accommodate for adverse effects as apparent from aberrant liver function tests (LFTs). In a specific embodiment when aspartate aminotransferase (AST) or alanine aminotransferase (ALT) increase to ≥3 times to 5 times the upper limit of normal (ULN), in particular when without signs of severe liver damage, dosage of compound 1 is lowered or interrupted for at least 2 weeks, e.g. daily dose of 600 mg is lowered to 400 mg, daily dose of 600 mg is lowered to 200 mg, or, daily dose of 200 mg is lowered to 100 mg, or, daily dose of 600 mg or 200 mg is discontinued for at least 2 weeks. In a further specific embodiment when aspartate aminotransferase (AST) or alanine aminotransferase (ALT) increase to ≥5 times to 8 times the upper limit of normal (ULN), in particular when without signs of severe liver damage, dosage of compound 1 is interrupted for at least 2 weeks, e.g. daily dose of 600 mg or 200 mg is discontinued for at least 2 weeks. In a further embodiment, should the dosage have been lowered or administration have been discontinued, dosage of compound 1 may be re-escalated provided AST and ALT decreased to <3 times ULN. In specific embodiments, re-escalation is done in a stepwise manner whereby dosage is re-escalated to the higher dose level for at least 2 weeks, such as from discontinuation to 100 mg/day, from discontinuation to 200 mg/day, from 100 mg/day to 200 mg/day, from 200 mg/day to 400 mg/day, or, from 400 mg/day to 600 mg/day. Prior to further dose escalation from a first or second re-escalation step, LFTs are determined.

Transdermal doses are generally selected to provide similar or lower blood levels than are achieved using injection doses.

When used to prevent the onset of a condition, a compound of the invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

A compound of the invention can be administered as the sole active agent or it can be administered in combination with other therapeutic agents, including other compound of the inventions that demonstrate the same or a similar therapeutic activity and that are determined to be safe and efficacious for such combined administration. In a specific embodiment, co-administration of two (or more) agents allows for significantly lower doses of each to be used, thereby reducing the side effects seen.

In one embodiment, a compound of the invention or a pharmaceutical composition comprising a compound of the invention is administered as a medicament. In a specific embodiment, said pharmaceutical composition additionally comprises a further active ingredient.

In one embodiment, a compound of the invention is co-administered with another therapeutic agent for the treatment of a fibrotic disease, particular agents include, but are not limited to 5-methyl-1-phenyl-2-(1H)-pyridone (pirfenidone; Esbriet®); Nintedanib (Ofev® or Vargatef®); STX-100 (ClinicalTrials.gov Identifier NCT01371305), FG-3019 (ClinicalTrials.gov Identifier NCT01890265), Lebrikizumab (CAS n #953400-68-5); Tralokinumab (CAS n #1044515-88-9). In another particular embodiment, the further therapeutic agent for the treatment and/or prophylaxis of a fibrotic disease is an autotaxin (or ectonucleotide pyrophosphatase/phosphodiesterase 2 or NPP2 or ENPP2) inhibitor, examples of which are described in WO 2014/139882.

In one embodiment, a compound of the invention is co-administered with another therapeutic agent for the treatment of a disease involving inflammation, particular agents include, but are not limited to, immunoregulatory agents e.g. azathioprine, corticosteroids (e.g. prednisolone or dexamethasone), cyclophosphamide, cyclosporin A, tacrolimus, mycophenolate, mofetil, muromonab-CD3 (OKT3, e.g. Orthocolone®), ATG, aspirin, acetaminophen, ibuprofen, naproxen, and piroxicam.

In one embodiment, a compound of the invention is co-administered with another therapeutic agent for the treatment and/or prophylaxis of arthritis (e.g. rheumatoid arthritis), particular agents include but are not limited to analgesics, non-steroidal anti-inflammatory drugs (NSAIDS), steroids, synthetic DMARDS (for example but without limitation methotrexate, leflunomide, sulfasalazine, auranofin, sodium aurothiomalate, penicillamine, chloroquine, hydroxychloroquine, azathioprine, tofacitinib, baricitinib, fostamatinib, and cyclosporin), and biological DMARDS (for example but without limitation infliximab, etanercept, adalimumab, rituximab, and abatacept).

In one embodiment, a compound of the invention is co-administered with another therapeutic agent for the treatment and/or prophylaxis of proliferative disorders, particular agents include but are not limited to: methotrexate, leukovorin, adriamycin, prednisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. Iressa®, Tarceva™, Erbitux™), VEGF inhibitors (e.g. Avastin™), proteasome inhibitors (e.g. Velcade™), Glivec® and hsp90 inhibitors (e.g. 17-AAG). Additionally, the compound of the invention according to Formula I may be administered in combination with other therapies including, but not limited to, radiotherapy or surgery. In a specific embodiment the proliferative disorder is selected from cancer, myeloproliferative disease or leukaemia.

In one embodiment, a compound of the invention is co-administered with another therapeutic agent for the treatment and/or prophylaxis of autoimmune diseases, particular agents include but are not limited to: glucocorticoids, cytostatic agents (e.g. purine analogs), alkylating agents, (e.g nitrogen mustards (cyclophosphamide), nitrosoureas, platinum compound of the inventions, and others), antimetabolites (e.g. methotrexate, azathioprine and mercaptopurine), cytotoxic antibiotics (e.g. dactinomycin anthracyclines, mitomycin C, bleomycin, and mithramycin), antibodies (e.g. anti-CD20, anti-CD25 or anti-CD3 (OTK3) monoclonal antibodies, Atgam® and Thymoglobuline®), cyclosporin, tacrolimus, rapamycin (sirolimus), interferons (e.g. IFN-β), TNF binding proteins (e.g. infliximab, etanercept, or adalimumab), mycophenolate, fingolimod and myriocin.

In one embodiment, a compound of the invention is co-administered with another therapeutic agent for the treatment and/or prophylaxis of transplant rejection, particular agents include but are not limited to: calcineurin inhibitors (e.g. cyclosporin or tacrolimus (FK506)), mTOR inhibitors (e.g. sirolimus, everolimus), anti-proliferatives (e.g. azathioprine, mycophenolic acid), corticosteroids (e.g. prednisolone, hydrocortisone), antibodies (e.g. monoclonal anti-IL-2Rα receptor antibodies, basiliximab, daclizumab), polyclonal anti-T-cell antibodies (e.g. anti-thymocyte globulin (ATG), anti-lymphocyte globulin (ALG)).

In one embodiment, a compound of the invention is co-administered with another therapeutic agent for the treatment and/or prophylaxis of asthma and/or rhinitis and/or COPD, particular agents include but are not limited to: beta2-adrenoceptor agonists (e.g. salbutamol, levalbuterol, terbutaline and bitolterol), epinephrine (inhaled or tablets), anticholinergics (e.g. ipratropium bromide), glucocorticoids (oral or inhaled). Long-acting β2-agonists (e.g. salmeterol, formoterol, bambuterol, and sustained-release oral albuterol), combinations of inhaled steroids and long-acting bronchodilators (e.g. fluticasone/salmeterol, budesonide/formoterol), leukotriene antagonists and synthesis inhibitors (e.g. montelukast, zafirlukast and zileuton), inhibitors of mediator release (e.g. cromoglycate and ketotifen), biological regulators of IgE response (e.g. omalizumab), antihistamines (e.g. ceterizine, cinnarizine, fexofenadine) and vasoconstrictors (e.g. oxymethazoline, xylomethazoline, nafazoline and tramazoline).

Additionally, a compound of the invention may be administered in combination with emergency therapies for asthma and/or COPD, such therapies include oxygen or heliox administration, nebulized salbutamol or terbutaline (optionally combined with an anticholinergic (e.g. ipratropium), systemic steroids (oral or intravenous, e.g. prednisone, prednisolone, methylprednisolone, dexamethasone, or hydrocortisone), intravenous salbutamol, non-specific beta-agonists, injected or inhaled (e.g. epinephrine, isoetharine, isoproterenol, metaproterenol), anticholinergics (IV or nebulized, e.g. glycopyrrolate, atropine, ipratropium), methylxanthines (theophylline, aminophylline, bamiphylline), inhalation anesthetics that have a bronchodilatory effect (e.g. isoflurane, halothane, enflurane), ketamine and intravenous magnesium sulfate.

In one embodiment, a compound of the invention is co-administered with another therapeutic agent for the treatment and/or prophylaxis of inflammatory bowel disease (IBD), particular agents include but are not limited to: glucocorticoids (e.g. prednisone, budesonide) synthetic disease modifying, immunomodulatory agents (e.g. methotrexate, leflunomide, sulfasalazine, mesalazine, azathioprine, 6-mercaptopurine and cyclosporin) and biological disease modifying, immunomodulatory agents (infliximab, adalimumab, rituximab, and abatacept).

In one embodiment, a compound of the invention is co-administered with another therapeutic agent for the treatment and/or prophylaxis of SLE, particular agents include but are not limited to: human monoclonal antibodies (belimumab (Benlysta)), Disease-modifying antirheumatic drugs (DMARDs) such as antimalarials (e.g. plaquenil, hydroxychloroquine), immunosuppressants (e.g. methotrexate and azathioprine), cyclophosphamide and mycophenolic acid, immunosuppressive drugs and analgesics, such as nonsteroidal anti-inflammatory drugs, opiates (e.g. dextropropoxyphene and co-codamol), opioids (e.g. hydrocodone, oxycodone, MS Contin, or methadone) and the fentanyl duragesic transdermal patch.

In one embodiment, a compound of the invention is co-administered with another therapeutic agent for the treatment and/or prophylaxis of psoriasis, particular agents include but are not limited to: topical treatments such as bath solutions, moisturizers, medicated creams and ointments containing coal tar, dithranol (anthralin), corticosteroids like desoximetasone (Topicort™), fluocinonide, vitamin D3 analogues (for example, calcipotriol), argan oil and retinoids (etretinate, acitretin, tazarotene), systemic treatments such as methotrexate, cyclosporine, retinoids, tioguanine, hydroxyurea, sulfasalazine, mycophenolate mofetil, azathioprine, tacrolimus, fumaric acid esters or biologics such as Amevive™, Enbrel™, Humira™, Remicade™, Raptiva™ and ustekinumab (a IL-12 and IL-23 blocker). Additionally, a compound of the invention may be administered in combination with other therapies including, but not limited to phototherapy, or photochemotherapy (e.g. psoralen and ultraviolet A phototherapy (PUVA)).

In one embodiment, a compound of the invention is co-administered with another therapeutic agent for the treatment and/or prophylaxis of allergic reaction, particular agents include but are not limited to: antihistamines (e.g. cetirizine, diphenhydramine, fexofenadine, levocetirizine), glucocorticoids (e.g. prednisone, betamethasone, beclomethasone, dexamethasone), epinephrine, theophylline or anti-leukotrienes (e.g. montelukast or zafirlukast), anti-cholinergics and decongestants.

By co-administration is included any means of delivering two or more therapeutic agents to the patient as part of the same treatment regime, as will be apparent to the skilled person. Whilst the two or more agents may be administered simultaneously in a single formulation, i.e. as a single pharmaceutical composition, this is not essential. The agents may be administered in different formulations and at different times.

In a particular aspect, the compound of the invention, such as the compound of formula I specifically, is administered as the sole active ingredient, more in particular as the sole active ingredient for the treatment of IPF. In a further embodiment, the compound of formula I is administered as the sole active ingredient according to any of the methods described herein allowing for single agent method of treatment.

### Abbreviations

ABBREVIATIONS

| Abbreviation | Definition |
|---|---|
| IPF | Idiopathic pulmonary fibrosis |
| HRCT | high-resolution computed tomography |
| BALF | bronchoalveolar lavage fluid |
| SLB | surgical lung biopsy |
| EDV | early discontinuation visit |
| EOS | end of the study |
| ICF | Informed consent form |
| FSH | Follicle-stimulating hormone |
| FVC | Forced vital capacity |
| FEV₁ | forced expiratory volume in 1 second |
| SAE | Severe adverse events |
| ALT | Alanine aminotransferase |
| AST | Aspartate aminotransferase |
| ULN | Upper limit of normal |
| eCRF | Electronic case report form |
| ECG | electrocardiogram |
| DBP | diastolic blood pressure |
| SBP | systolic blood pressure |
| SGRQ | St. George's Respiratory Questionnaire |
| DLCO | diffusing capacity for the lungs for carbon monoxide |
| FRI | functional respiratory imaging |
| mSv | millisieverts |
| TLC | Total lung capacity |

### Chemical Synthetic Procedures

The compound of the invention can be prepared from readily available starting materials as described in WO 2014/139882 and WO 2014/202458.

### BIOLOGICAL EXAMPLES

### Example 1

### Clinical Study

1.1. Study Design

The study is a randomized, double-blind, parallel group, placebo-controlled, multicenter study to evaluate the test compound in subjects with IPF.

During the screening period, following signing of the informed consent form, the subject's historical chest HRCT and surgical lung biopsy (SLB) are sent to central review for confirmation of the IPF diagnosis.

At baseline, after the subject's eligibility for the study has been confirmed, subjects are randomized in a 3:1 ratio test compound (600 mg q.d.) : matching placebo administered for 12 weeks.

The subjects visit the clinical study center at screening (Day-28 to Day-4), Day-1 (baseline), Week 1 (Day 7), Week 2 (Day 14), Week 4 (Day 28), Week 8 (Day 56), and Week 12 (Day 84) or the early discontinuation visit (EDV). In addition, a follow-up visit is planned 2 weeks after the last administration of study drug (Week 98 [Day 98]).

Each subject is in the study for up to approximately 18 weeks (from screening to follow-up).

The end of the study (EOS) is defined as the last contact with the last subject in the study.

The assessments performed at each visit are detailed in the study flow chart (Section 1). To enhance the safety and integrity of the study data, an independent medical safety review is implemented. (See Section 7 for additional information).

1.2. Study Population

Approximately 24 subjects with a centrally confirmed IPF diagnosis are randomized.

TABLE I
Baseline patient demographic parameters

| | | Compound | |
|---|---|---|---|
| | | according | |
| Baseline | Placebo | to Formula I | Total |
| demographics | (N = 6) | (N = 17) | (N = 23) |
| Males (%) | 83 | 59 | 65 |
| Age (mean, yrs) | 62.5 | 66.6 | 65.6 |
| Race (%) | | | |
| White | 100 | 100 | 100 |
| BMI (mean, kg/m²) | 32.40 | 29.35 | 30.15 |
| Smokers (%) | | | |
| Former | 50 | 35 | 39 |
| Never | 50 | 65 | 61 |
| Duration of IPF | 1.0 | 1.9 | 1.7 |
| (mean; yrs) | | | |
| DLCO | 40.6 | 37.8 | 38.6 |
| (mean, % predicted of normal) | | | |
| Baseline FVC | 2.693 | 2.777 | 2.755 |
| (mean, L) | | | |
| Baseline FVC | 69.7 | 75.3 | 73.8 |
| (mean, predicted) | | | |

1.2.1. Inclusion Criteria

Subjects who meet all of the following criteria are eligible for the study:
1. Subjects who are able and willing to sign the ICF as approved by the independent ethics committee (IEC).
2. Male or female subjects of non-child-bearing potential aged ≥40 years on the day of signing the ICF.
Note: Female subjects will be considered of non-childbearing potential if they are either sterilized, ovariectomized, hysterectomized, or postmenopausal (i.e., at least 24 months of amenorrhea in the absence of other biological or physiological causes [in case of doubt, the subject's follicle stimulating hormone [FSH] levels will be determined and the subject will be considered postmenopausal if the FSH level is ≥35 mIU/mL).
3. Subjects with a chest HRCT performed within 12 months prior to screening visit.
4. Subjects with IPF diagnosed by a multidisciplinary team and confirmed by central review of the subject's HRCT pattern and SLB (if available) (see Appendix 2 for additional details).
5. Subjects meeting all of the following criteria:

a. FVC≥50% predicted of normal

b. Diffusing capacity for the lungs for carbon monoxide (DLCO)≥30% predicted of normal (corrected for hemoglobin) (see Appendix 3 for additional details).
6. Subjects with a forced expiratory volume in 1 second (FEV1)/FVC (Tiffeneau-Pinelli index) ratio≥0.70 (based on pre-bronchodilator spirometry).
7. Subjects who are on stable supportive care (e.g., supplemental oxygen, pulmonary rehabilitation) for at least 3 weeks prior to screening and during screening period.
8. Subjects must be in a stable condition and acceptable for study participation based upon the results of a medical history, physical examination, vital signs, 12-lead ECG, and laboratory evaluation.
9. Subjects must have an estimated minimum life expectancy of 12 months in the opinion of the investigator.
10. Male subjects and their female partners must use a highly effective method of birth control. Additional details on these methods are provided in Section 4.2.4.
11. Subjects who are able to understand the importance of adherence to study treatment, study procedures and requirements, including the concomitant medication restrictions.

1.2.2. Exclusion Criteria

Subjects meeting one or more of the following criteria cannot be selected for this study:
1. Subjects with a known hypersensitivity to any of the study drug ingredients or a history of a significant allergic reaction to any drug as determined by the investigator (e.g., anaphylaxis requiring hospitalization).
2. Subjects with a history of or a current immunosuppressive condition (e.g., human immunodeficiency virus [HIV] infection).
3. Subjects with a history of malignancy within the past 5 years (except for carcinoma in situ of the uterine cervix, basal cell carcinoma of the skin that has been treated with no evidence of recurrence, and prostate cancer medically managed through active surveillance or watchful waiting).
4. Subjects with clinically significant abnormalities detected on ECG regarding either rhythm or conduction (e.g., QT interval corrected for heart rate using Fridericia's formula [QTcF]≥450 ms, or a known long QT syndrome).
Note: A first degree heart block will not be considered as a significant abnormality.
5. Subjects with acute IPF exacerbation within 6 weeks prior to screening and during the screening period.
6. Subject with a lower respiratory tract infection requiring antibiotics within 4 weeks prior to screening and during the screening period.
7. Subjects who have been smoking within 3 months prior to screening.
8. Intersitial lung disease (ILD) associated with known primary diseases (e.g., sarcoidosis, amyloidosis, etc.), exposures (e.g., radiation, silica, asbestos, coal dust, etc.), and drugs (e.g., amiodarone, etc.).
9. Subjects with a history of lung volume reduction surgery or lung transplant.
10. Subjects with an unstable cardiac or pulmonary disease (other than IPF) within 6 months prior to screening or during the screening period, including but not limited to:

a. unstable angina pectoris, myocardial infarction

b. congestive heart failure requiring hospitalization
11. Subjects with any clinical condition or circumstance that in the opinion of the investigator may make a subject unsuitable for inclusion or unable to complete the study or comply with study procedures and requirements.
12. Subjects with a contra-indication for bronchoscopy and bronchoalveolar lavage in the opinion of the investigator.
13. Subjects with an abnormal liver function defined as aspartate aminotransferase (AST), alanine aminotransferase (ALT), or bilirubin >3 x upper limit of the normal range (ULN).
14. Subjects with an abnormal renal function defined as creatinine clearance<50 mL/min using the Cockroft-Gault equation.
15. Subjects participating in a drug/device or biologic investigational research study (concurrently with the current study or within 8 weeks prior to screening).
16. Subjects using of any of the following therapies within 4 weeks before screening:

a. Pirfenidone

b. Nintedanib

c. Warfarin

d. Imatinib

e. Ambrisentan

f. Azathioprine

g. Cyclophosphamide

h. Cyclosporine A

i. Prednisone at steady dose>15 mg/day (for details, see Section 4.2.4)

j. Any experimental IPF therapy
17. Subjects with active alcohol or substance abuse in the opinion of the investigator.

1.2.3. Prohibitions and Restrictions

Smoking is forbidden at all times (from 3 months before screening to the end of the study).

The following therapies are not allowed during the course of the study (and within 4 weeks prior to screening): pirfenidone, nintedanib, warfarin, imatinib, ambrisentan, azathioprine, cyclophosphamide, cyclosporine A, prednisone at steady dose>15 mg/day (i.e., the use of prednisone is precluded unless subjects have been on a stable dose≤15 mg daily and it is anticipated that they will continue on this stable dose for the duration of the study), and any experimental IPF therapy.

Male subjects and their female partners must use 2 generally accepted adequate contraceptive methods, 1 of which is a barrier method (e.g., condom in combination with hormonal contraception stabilized for at least 1 month) from screening until 3 months after the study. In addition, sperm donation is not allowed until 3 months after the last study visit. In a case where the female partner of a male subject has undergone documented surgical sterilization that was performed more than 1 year before screening, the subject is not required to use an additional form of contraception.

1.2.4. Removal of Subjects from Therapy or Assessments

A subject may be discontinued from the study at any time without the subject's consent if the investigator or sponsor determines that it is not in the best interest of the subject to continue participation.

Subjects may withdraw from the study at any time, for any reason, without jeopardizing their clinical care.

Subjects may be withdrawn from the study for any of the following reasons:
noncompliance with the study drug
noncompliance with the study procedures
lost to follow-up
SAEs or severe AEs can be considered a reason for discontinuation of treatment, preferably after consultation with the medical monitor
investigator request
sponsor request

A subject may be withdrawn from the study after discussion between the investigator and the medical monitor for any of the following reasons:
use of concurrent therapy that was not permitted
prolonged interruption of study drug (i.e., interruption>7 days)
life-threatening AE or an SAE that places the subject at immediate risk
increase in liver function tests (LFTs) to 3×ULN (if the baseline LFTs are normal) or an increase that exceeds an absolute value of 5×ULN (regardless of whether baseline LFTs are normal)

A subject must be withdrawn from the study for any of the following reasons:
serious infections (those requiring parenteral antimicrobial therapy and/or hospitalization) arrhythmia or conduction abnormality (including but not limited to prolonged QTcF, where the severity is categorized as Common Terminology Criteria for Adverse Events [CTCAE] grade 3 or higher)
clinical laboratory test results, which are determined by the investigator in consultation with the medical monitor to be clinically significant and require discontinuation of study drug; changes in LFTs are defined as follows (confirmed upon repeat testing):
elevated ALT or AST of >8×ULN
elevated ALT or AST of >5×ULN for >2 weeks
total bilirubin>2×ULN and/or clinical jaundice, in association with elevation of ALT or AST>3×ULN
no convincing alternative etiology (e.g., viral hepatitis, alcohol ingestion, trauma) for the elevated transaminase is identified, regardless of whether ALT or AST levels had improved
unblinding of a subject's study treatment assignment to the subject, the site staff, or the blinded sponsor staff while on study drug
close of the study by the sponsor or regulatory authorities
wish of the subject to withdraw (At any time and for any reason, a subject's participation in the study may terminate at his/her request without prejudice to his/her future medical care.)
worsening of the subject's disease condition, which in the investigator's opinion needs an alternative treatment approach

In case clinically significant laboratory test results are a potential reason for discontinuation from the study drug and withdrawal from the study, retesting should be prompted (within 3 to 5 days) after the investigator has consulted with the medical monitor. A decision regarding subject discontinuation should be made only after the results from the retest are available.

Subjects who stop study drug for any reason will not be replaced. Subjects withdrawing from the study will be encouraged to complete the same final evaluations as subjects completing the study according to the protocol, particularly safety evaluations in the subject's interest so that data can be recorded in the same way as for subjects who completed the study. The reason(s) for withdrawal will be documented in the electronic case report form (eCRF). Note that randomized subjects who drop out before the first administration of the study drug will be replaced.

Reasonable efforts (3 attempts) will be made to get confirmation on the reasons for dropout for subjects who are lost to follow-up. These attempts must be documented in the subject's file.

The sponsor has the right to terminate the study at any time in case of safety concerns or if special circumstances concerning the study drug or the company itself occur, making further treatment of subjects impossible. In this event, the investigator(s) and relevant authorities will be informed of the reason for study termination.

1.3. Study Protocol

1.3.1. Dosage and Administration

Subjects receive 600 mg of test compound (3 capsules of 200 mg) or placebo q.d. for 12 weeks administered in the morning.

When dosing takes place at the clinical study center, a volume of 240 mL water will be provided to each subject to be consumed immediately and completely at the time of dosing. Subjects will be instructed to swallow the study drug whole, and not chew the drug prior to swallowing.

If a subject misses a dose (e.g., because he/she forgot to take the medication), he/she should take the missed dose within 12 h after the planned intake time. If the study drug is not taken within 12 h after the planned time, the missed dose should be skipped.

Dose changes during the study are not allowed. Instead, the subject should either temporarily stop all intake or permanently stop study drug.

1.3.2. Prior and Concomitant Therapy

Should any treatment other than the study drug be used during the course of the study, the name of the drug, the dosage, the route, and the dates (and time) of administration must be recorded in the eCRF system. Prior and concomitant medications (taken up to 8 weeks prior to Day 1) will be recorded from the study inclusion date (ICF signed) until the last visit.

Concomitant therapies taken for the long-term treatment of preexisting conditions can continue during the study provided they are in accordance with the inclusion and exclusion criteria (see above). It is required that these medications be stabilized prior to study entry and continued without variation of dose or regimen during the study.

In a case additional concomitant medication needs to be administered or dose adjustments for preexisting conditions need to be performed during the study, the risk/benefit to the subject should be carefully assessed and consideration given to the timing of any necessary introduction of new medications.

If the subject shows a worsening of his/her IPF disease condition (acute IPF exacerbation), all treatment options are allowed at the investigator's discretion. The decision to continue treatment with study drug should be taken on a case-by-case basis, preferably after discussion with the study's medical monitor.

1.4. Study Assessments

The study assessments as described below is performed at the following time points:
Visit 1: D-28 to D-4, Screening
Visit 2: D-1, baseline
Visit 3: D7±2 days
Visit 4: D14±2 days
Visit 5: D28±3 days
Visit 6: D56±3 days
Visit 7: D84±3 days
Visit 8/EOS: D98±3 days

Visits are scheduled in such a way that the total study duration from baseline to last dosing does not exceed 13 weeks.

The sequence of study assessments, will be as follows (if applicable):
1. physical examination, oral body temperature, ECG, systolic and diastolic blood pressure (SBP and DBP), supine heart rate, respiratory rate
2. SGRQ
3. assessment of (S)AE(s) and concomitant medication
4. blood sampling for PK
5. blood sampling for safety laboratory analysis
6. blood sampling for PD
7. blood sampling for biomarkers
8. spirometry
9. DLCO
10. HRCT-scan
11. bronchoscopy (biomarkers and PD in BALF)

1.5. Subject Self-Assessment

Subjects will be given a diary card at Day-1 (baseline) to record the following:
From Day 1 through Day 84 (or the EDV), subjects will be asked to record the date (and time) of study drug intake and the number of capsules taken for each administration.
From Day 1 to Day 84 (or the EDV), subjects will be asked to indicate whether a home based spirometry test was done and if no such test was done, the reason(s) why should be provided.
From Day 1 to Day 84 (or the EDV), subjects will be asked to record changes in concomitant medication regimen, including new medicines not captured in medication history, use of bronchodilators, any other concomitant medication used as well as any emerging AE.

Subjects will be instructed to bring the diary card and used/unused study drug to each visit. All diary card data will be entered in the eCRF by the designated site personnel.

1.6. Efficacy Assessment
1.6.1. Pulmonary Function by Spirometry

Pulmonary function will be assessed through spirometry both performed at the study center (at screening, baseline, and thereafter post dose at the time points indicated in the study schedule and according to sequence of study assessments) and at home (at baseline and thereafter post dose; on a daily basis in the morning). Specific instructions on how to perform the spirometry at home are provided in a separate spirometry user manual.

The site-based spirometry must meet the criteria for acceptability and repeatability as defined in the ATS/ERS guidelines (Miller et al., 2005)

Pulmonary function will be measured in standardized manner, results will be transmitted electronically and confirmed by a central reader.

1.6.1.1. Timing of Home- and Site-Based Spirometry

The spirometry test is to be performed in the morning, preferably at approximately the same time (±1 h) every day/visit.

1.6.1.2. Timing of Home- and Site-Based Spirometry Versus Bronchodilator Use

All spirometry evaluations should be performed pre-bronchodilator. Pre-bronchodilator spirometry is defined as spirometry testing performed for a subject who has:
withheld their short-acting β-agonist (e.g., albuterol) or anticholinergic (e.g., ipratropium bromide) for >6 h prior to the spirometry assessment AND
withheld their long-acting bronchodilator (e.g., salmeterol, formoterol) for ≥12 h and other longer-acting agents (e.g., indacaterol, tiotropium) for ≥24 h prior to the spirometry assessment

In case the subject is on bronchodilators, he/she can use the bronchodilator after the spirometry but prior to HRCT for FRI parameters.

1.6.1.3. Spirometry Parameters and Calculation of Predicted Values

The following parameters will be measured as part of the spirometry assessment:
FEV1 (L) and percent predicted FEV1
FVC (L) and percent predicted FVC
FEV1/FVC ratio
Forced expiratory flow (FEF) between 25 and 75% of exhaled volume (FEF25-75)

The ‘2012 Global Lung Function Initiative Equations’ will be used to estimate the predicted values. (Quanjer et al., 2012)

TABLE II
Study FVC values

| | Week 4 | | Week 8 | | Week 12 | | Follow up | |
|---|---|---|---|---|---|---|---|---|
| Time point | Placebo | Cpd I | Placebo | Cpd I | Placebo | Cpd I | Placebo | Cpd I |
| N# subjects/baseline | 3/6 | 16/17 | 4/6 | 15/17 | 4/6 | 13/17 | 4/7 | 15/17 |
| FVC CFB (mL) | −87 | +116 | −140 | +15 | −87 | +8 | −205 | −55 |

| | | | | |
|---|---|---|---|---|
| p-value CFB | — | p < 0.05 | — | — |

Over the 12-week period, subjects receiving the compound according to Formula I showed an FVC increase of 8 mL (mean from baseline), whereas those on placebo showed an FVC reduction of 88 mL (mean from baseline).

1.7. Biomarker Analysis

1.7.1. Blood Samples

Blood samples of 14 mL will be collected predose by venipuncture (or indwelling catheter for pharmacokinetic sampling days) in the arm at the time points indicated in the study flow chart and according to sequence of study assessments.

From the blood samples, the LPA 18:2 levels are determined using laboratory techniques. The percentage reduction in LPA18:2 levels is presented in the table below.

TABLE III
LPA18:2 percentage reduction during the study

| Time point | Placebo (n# subjects) | | Compound I (n# subjects) | |
|---|---|---|---|---|
| Baseline | — | | — | |
| Week 4 | 14.23% | (N = 5) | 52.48% | (N = 16) |
| Week 12 | 2.29% | (N = 6) ** | 67.48% | (N = 15) |
| Follow up visit | −0.27% | (N = 5) | −34.36% | (N = 15) |

NB:
a positive value means a reduction in LPA levels
a negative value means an increase in LPA levels
* p < 0.05
** p < 0.01
*** p < 0.005

The results indicate that administering the compound I results in a stable decrease in LPA 18:2 levels, whereas stopping the administration results in levels returning to or above baseline levels.

The test compound plasma levels were determined by LC-MS/MS and PK parameters by non-compartmental analysis using Phoenix WinNonlin.

At week 4, the median maximum observed plasma concentration (Cₘₐₓ) of compound I was 6.06 μg/mL reached at a median tₘₐₓ of 4 h. The mean area under the plasma concentration time curve for the dosing interval of 24 hours (AUC_{0-τ}) was 55.6 μg.h/mL associated with a trough plasma concentration (C_{τ}) of 604 ng/mL being around 4-fold higher than the IC₅₀ in human plasma assay for the reduction of LPA 18:2 (143 ng/mL).

A similar PK/PD profile was observed in IPF patients of the current study compared to healthy volunteers (as illustrated in Table IV.

TABLE IV
PK/PD correlation in plasma

| | | Compound I concentration | | LPA C8:2 |
|---|---|---|---|---|
| Population | Time point | nM | ng/mL | % reduction |
| Healthy volunteers | D 14 - 24 h post-dose | 421 | 249 | 66 |
| | D 14 - 6 h post-dose | 6506 | 3830 | 83 |
| IPF patients | Week 4 - 0 h post-dose | 1020 | 604 | 52 |
| | Week 12 - 0 h post-dose | 1698 | 1000 | 68 |
| | Week 4 - 0 h post-dose | 6557 | 3860 | 88 |

1.7.2. Biomarkers

The following exploratory biomarkers are evaluated in blood samples:
KL-6/Muc1
surfactant protein A and D
CCL18
ATX
MMP1, MMP7
markers of extracellular matrix (ECM) turnover (neoepitope assay)

A statistically significant increase is seen in surfactant protein A in blood following 12 weeks of treatment with Compound 1.

TABLE V
Biomarker analysis surfactant protein A in blood following 12 weeks treatment

| | Placebo q.d. | | | | | Compound I 600 mg q.d. | | | | | |
|---|---|---|---|---|---|---|---|---|---|---|---|
| | n = 6 | | | | | n = 17 | | | | | |
| | | | | Mean | | | | | Mean | | |
| | | | Mean | % | | | | Mean | % | | |
| | | Mean | CFB | Change | | | Mean | CFB | Change | | p value |
| | | (95% | (95% | (95% | | | (95% | (95% | (95% | | mean |
| | n¹ | CI) | CI) | CI) | p value² | n¹ | CI) | CI) | CI) | p value² | CFB³ |
| Baseline | 6 | 513 · 8 | | | | 17 | 416 · 9 | | | | |
| | | (−13 · 8, | | | | | (294 · 5, | | | | |
| | | 1041 · 5) | | | | | 539 · 3) | | | | |
| Week 4 | 5 | 575 · 1 | −9 · 8 | −3 · 8 | 0 · 8599 | 14 | 490 · 8 | 104 · 2 | 45 · 5 | 0 · 0242 | 0 · 7789 |
| | | (−118 · 6, | (−153 · 7, | (−38 · 3, | | | (359 · 0, | (15 · 9, | (9 · 7, | | |
| | | 1268 · 9) | 134 · 2) | 30 · 7) | | | 622 · 5) | 192 · 6) | 81 · 3) | | |
| Week 12 | 5 | 349 · 8 | 36 · 6 | 18 · 7 | 0 · 1214 | 14 | 593 · 6 | 179 · 2 | 57 · 2 | 0 · 0213 | 0 · 0305 |
| | | (247 · 2, | (−15 · 2, | (−12 · 5, | | | (366 · 1, | (31 · 3, | (18 · 7, | | |
| | | 452 · 4) | 88 · 5) | 49 · 9) | | | 821 · 2) | 327 · 0) | 95 · 7) | | |
| Follow-up | 5 | 606 · 3 | 21 · 4 | 1 · 5 | 0 · 3641 | 15 | 532 · 1 | 94 · 6 | 29 · 1 | 0 · 1878 | 0 · 0234 |
| | | (−104 · 9, | (−36 · 6, | (−3 · 6, | | | (324 · 5, | (−51 · 9, | (1 · 8, | | |
| | | 1317 · 4) | 79 · 4) | 6 · 6) | | | 739 · 7) | 241 · 2) | 56 · 3) | | |

¹n = number of samples used to calculate mean absolute values.
²Within-group paired t-test versus baseline.
³Type III p values for treatment effects. All from ANCOVA model.

The following additional biomarkers may be analyzed in blood samples if deemed appropriate after results of the above exploratory markers have become available:
oxidative stress: ICAM-1 and VCAM-1
neutrophil recruitment, activation: IL8, S100A12
other biomarkers might be analyzed if deemed appropriate (e.g., serum protein, serum miRNA)

ATX will be determined in the supernatant of the BALF. Broncho alveolar lavage (BAL) cell count will be performed. BAL cell pellets will be stored for possible future analyses (transcriptomics, proteomics).

1.8. Functional Respiratory Imaging (FRI)

HRCT will be performed (post dose if applicable) at the time points indicated in the study chart (see 1.4 above) and according to sequence of study assessments. HRCT scans will be used to generate FRI measurements allowing for an evaluation of regional IPF disease manifestation and disease progression. In case the subject is on bronchodilators, he/she can use the bronchodilator after the spirometry but prior to HRCT for FRI parameters.

At each computed tomography (CT) assessment an inspiratory and an expiratory scan will be taken. During the baseline visit an additional scan of the upper airway will be taken. The inspiratory and an expiratory scan will expose the subject to a total radiation dose of approximately 4-5 mSv per visit, including the initial CT localizer radiograph (topogram scout). The upper airway scan will expose a subject to approximately 1 mSv. A radiation dose of approximately 2-2.5 mSv is equivalent to approximately 1 year of background radiation (based on the assumption of an average “effective dose” from natural background radiation is of 3.1 mSv per year in the US and 2.4 mSv per year in Europe. (USNRC, 2014; WNA, 2015)

All CT images will be imported into Mimics, a commercial, medical image processing software package (Materialise, Leuven, Belgium, Food and Drug Administration, K073468; CE certificate, BE 05/1191 CE01) for analysis. This software package converts the HRCT images into patient specific, 3-dimensional computer models of the lung lobes, the airway lumen and wall, and the vascular tree. The airway and vascular tree are evaluated at functional residual capacity (FRC) and total lung capacity (TLC) level and can be segmented down to bronchi/vessels with a diameter of around 1-2 mm. Beyond this point the HRCT resolution is insufficient to distinguish alveolar and intraluminal air, or blood vessel tissue and surrounding lung tissues. A typical airway model includes 5-10 generations, depending mainly on the disease state of the individual patient. Afterwards the airway lumen models will be processed further to obtain a model that is suitable for flow calculations.

The following FRI parameters based on low-dose inspiratory-expiratory multi-slice CT images and computational fluid dynamics flow simulations will be evaluated.
lobar volumes at FRC and TLC
airway volumes at FRC and TLC
airway resistance
internal lobar airflow distribution
low attenuation or emphysema score at TLC
blood vessel density or fibrosis score at TLC
airway wall thickness at TLC
air trapping at FRC
mass of deposited particles per defined airway section

Following the protocol above the following results have been obtained:

TABLE VI
Specific airway volume (mL/L)

| | Baseline | Week 12 |
|---|---|---|
| Placebo | 4.45 | 5.24 |
| Compound 1 | 3.92 | 3.80 |

TABLE VII
Specific airway resistance (kPa/sec)

| | Baseline | Week 12 |
|---|---|---|
| Placebo | 0.10 | 0.06 |
| Compound 1 | 0.11 | 0.12 |

The results of table VI and VII above show the stabilization of the airway volume and resistance in subjects receiving Compound 1.

### Example 2

### Population Pharmacokinetic (PK) and Pharmacodynamics (PD) Analysis

The aim of the current example is to describe the exposure response relationship of compound 1. To that end, the PK data and LPA 18:2 response upon administration of compound I in healthy volunteers and IPF patients in three clinical trials are subjected to a combined population PK and PK/PD model. Two trials are conducted in healthy volunteers. The third trial is conducted in IPF patients and is described in example 1 above.

A first trial is a randomized, double-blind, placebo-controlled, single center study evaluating single ascending doses (SAD) and multiple ascending (MAD) doses of compound 1 in healthy male volunteers (see Van der Aar E M, 2016).

In a second trial, interaction of rifampicin with 600 mg of compound 1 is evaluated. This is an open-label, single center drug-drug interaction (DDI) study evaluating the effect of repeated doses of rifampin (600-mg capsule) for 10 days on the single-dose PK of compound 1 (600-mg capsule) in 18 healthy male volunteers. Blood samples are collected for PK assessment before and after repeated dosing with rifampin.

The population PK analysis and statistical methods used are based on the food and Drug Administration and European Medicines Agency guidance document. The exposure response (autotaxin inhibition) relationship of compound 1 is first described using non-linear mixed-effects modelling and the model is subsequently deployed to simulate LPA 18:2 reduction as biomarker of autotaxin activity in the dose range of 50 to 1000 mg of compound 1 once or twice daily. Dose, formulation, rifampicin co-administration, health status (healthy volunteer versus IPF patient) and baseline LPA 18:2 are identified as covariates in the model. The effect of dose on systemic clearance indicates that compound 1 follows a more than dose-proportional PK over the dose range of 50 to 1000 mg once daily. Model-based simulations show a reduction of LPA 18:2 of at least 80% with doses greater or equal to 200 mg once daily.

Table VII shows the simulated AUC, AUE, Cmax, and maximal plasma LPA 18:2 reduction (with 95% CI) at steady state for compound 1 across a range of dosing regimens between 50 mg QD and 1000 mg BID for the typical IPF patient. The simulations showed that a dose of 200 mg QD led to 81% (79-83%) LPA 18:2 reduction and at a dose of 600 mg QD, the expected reduction was 88% (86-89%).

TABLE VIII
Table VII Simulated typical patient dose-response relation of compound 1 pharmacokinetics
and plasma lysophosphatidic acid 18:2 pharmacodynamics at steady state [95% CI]

| Cpd I | | | | | Maximal plasma | | | |
|---|---|---|---|---|---|---|---|---|
| treatment | Cpd I Cₘₐₓ | | Cpd I AUC | | LPA 18:2 | | Plasma LPA 18:2 | |
| (mg total | (ng/mL) | | (mcg/mL · hr) | | reduction (%) | | AUE (% · hr) | |
| daily dose) | [95% CI] | | [95% CI] | | [95% CI] | | [95% CI] | |
| 50 QD | 204 | [172; 254] | 2.19 | [1.96; 2.58] | 60.7 | [55.2; 66.6] | 1010 | [902; 1140] |
| 50 BID | 124 | [108; 147] | 2.19 | [1.96; 2.58] | 51.8 | [46.8; 57.8] | 1100 | [970; 1240] |
| 100 QD | 413 | [348; 514] | 4.48 | [4.02; 5.25] | 73 | [68.9; 76.9] | 1360 | [1240; 1480] |
| 100 BID | 252 | [220; 299] | 4.48 | [4.02; 5.25] | 66.3 | [62; 70.6] | 1470 | [1360; 1580] |
| 150 QD | 626 | [528; 780] | 6.86 | [6.17; 8.02] | 78.2 | [75.1; 81] | 1550 | [1440; 1660] |
| 150 BID | 385 | [336; 457] | 6.86 | [6.17; 8.02] | 73.1 | [69.5; 76.5] | 1650 | [1570; 1750] |
| 200 QD | 845 | [713; 1050] | 9.36 | [8.43; 10.9] | 81.1 | [78.7; 83.3] | 1670 | [1570; 1770] |
| 200 BID | 522 | [456; 620] | 9.36 | [8.43; 10.9] | 77 | [74.1; 79.8] | 1770 | [1690; 1840] |
| 300 QD | 1300 | [1100; 1610] | 14.7 | [13.3; 16.9] | 84.3 | [82.4; 86] | 1820 | [1740; 1890] |
| 300 BID | 811 | [711; 962] | 14.7 | [13.3; 16.9] | 81.4 | [79.3; 83.4] | 1900 | [1840; 1950] |
| 600 QD | 2830 | [2370; 3480] | 34.3 | [31.1; 38.5] | 87.7 | [86.3; 89.2] | 2020 | [1950; 2050] |
| 600 BID | 1830 | [1610; 2150] | 34.3 | [31.1; 38.5] | 86.4 | [84.9; 87.9] | 2050 | [2010; 2090] |
| 1000 QD | 5420 | [4460; 6690] | 73.3 | [63.4; 84.2] | 89.2 | [87.6; 90.8] | 2110 | [2050; 2140] |
| 1000 BID | 3720 | [3150; 4390] | 73.3 | [63.4; 84.2] | 88.6 | [87; 90.2] | 2120 | [2080; 2160] |

Abbreviations:
AUC = area under the curve,
AUE = area under the effect curve,
BID = twice daily,
Cₘₐₓ = maximum plasma concentration,
QD = once daily,
LPA = lysophosphatidic acid

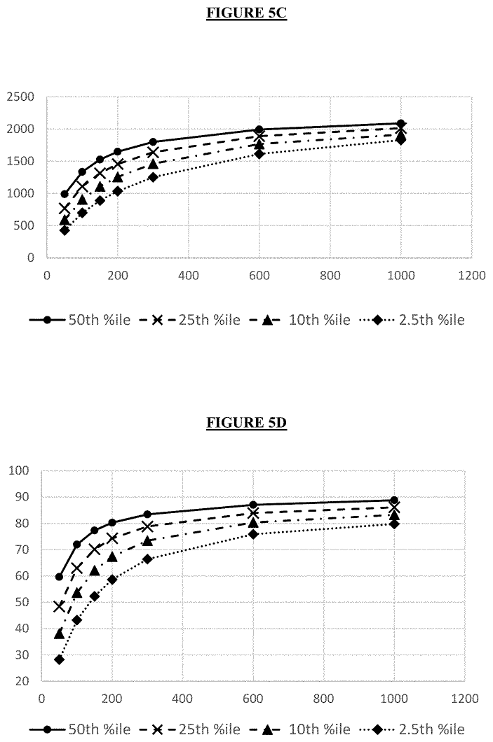

FIG. 5 shows visualizations of the 50th, 25th, 10th, and 2.5th percentiles of individual simulated compound 1 Cₘₐₓ and AUC, and maximal plasma LPA 18:2 reduction and AUEC within the 50- to 1000-mg QD dose range. The impact of between-patient variability is illustrated by the difference between the curves representing different percentiles of the simulated population in each panel. There is an increasing response when increasing the dose from 200 to 300 and 600 mg QD for all summary variables. Most simulated patients reached 50% inhibition at 200 mg QD, with limited improvement in this metric when increasing the dose to 600 mg QD.

### Final Remarks

It will be appreciated by those skilled in the art that the foregoing descriptions are exemplary and explanatory in nature, and intended to illustrate the invention and its preferred embodiments. Through routine experimentation, an artisan will recognize apparent modifications and variations that may be made without departing from the spirit of the invention. All such modifications coming within the scope of the appended claims are intended to be included therein. Thus, the invention is intended to be defined not by the above description, but by the following claims and their equivalents.

All publications, including but not limited to patents and patent applications, cited in this specification are herein incorporated by reference as if each individual publication are specifically and individually indicated to be incorporated by reference herein as though fully set forth.

It should be understood that factors such as the differential cell penetration capacity of the various compounds can contribute to discrepancies between the activity of the compounds in the in vitro biochemical and cellular assays.

At least some of the chemical names of compound of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

### REFERENCES

King, T. E. J., Bradford, W. Z., Castro-Bernardini, S., Fagan, E. A., Glaspole, I., Glassberg, M. K., Gonna, E., Hopkins, P. M., Kardatzke, D., Lancaster, L., Lederer, D. J., Nathan, S. D., Pereira, C. A., Sahn, S. A., Sussman, R., Swigris, J. J., Noble, P. W., 2014. A Phase 3 Trial of Pirfenidone in Patients with Idiopathic Pulmonary Fibrosis. N. Engl. J. Med. 370, 2083-2092. doi:10.1056/NEJMoa1402582
Ley, B., Collard, H. R., King, T. E., 2011. Clinical Course and Prediction of Survival in Idiopathic Pulmonary Fibrosis. Am. J. Respir. Crit. Care Med. 183, 431-440. doi:10.1164/rccm.201006-0894CI
Meltzer, E. B., Noble, P. W., 2008. Idiopathic pulmonary fibrosis. Orphanet J. Rare Dis. 3, 8. doi:10.1186/1750-1172-3-8
Miller, M. R., Hankinson, J., Brusasco, V., Burgos, F., Casaburi, R., Coates, A., Crapo, R., Enright, P., van der Grinten, C. P. M., Gustafsson, P., Jensen, R., Johnson, D. C., Maclntyre, N., McKay, R., Navajas, D., Pedersen, O. F., Pellegrino, R., Viegi, G., Wanger, J., ATS/ERS Task Force, 2005. Standardisation of spirometry. Eur. Respir. J. 26, 319-338. doi:10.1183/09031936.05.00034805
Noble, P. W., Albera, C., Bradford, W. Z., Costabel, U., Glassberg, M. K., Kardatzke, D., King, T. E., Lancaster, L., Sahn, S. A., Szwarcberg, J., Valeyre, D., du Bois, R. M., 2011. Pirfenidone in patients with idiopathic pulmonary fibrosis (CAPACITY): two randomised trials. The Lancet 377, 1760-1769. doi:10.1016/S0140-6736(11)60405-4
Pérez, E. R. F., Daniels, C. E., Schroeder, D. R., St. Sauver, J., Hartman, T. E., Bartholmai, B. J., Yi, E. S., Ryu, J. H., 2010. Incidence, Prevalence, and Clinical Course of Idiopathic Pulmonary Fibrosis. Chest 137, 129-137. doi:10.1378/chest.09-1002
Quanjer, P. H., Stanojevic, S., Cole, T. J., Baur, X., Hall, G. L., Culver, B. H., Enright, P. L., Hankinson, J. L., Ip, M. S. M., Zheng, J., Stocks, J., 2012. MULTI-ETHNIC REFERENCE VALUES FOR SPIROMETRY FOR THE 3-95 YEAR AGE RANGE: THE GLOBAL LUNG FUNCTION 2012 EQUATIONS. Eur. Respir. J. 40, 1324-1343. doi:10.1183/09031936.00080312
Raghu, G., Collard, H. R., Egan, J. J., Martinez, F. J., Behr, J., Brown, K. K., Colby, T. V., Cordier, J.-F., Flaherty, K. R., Lasky, J. A., Lynch, D. A., Ryu, J. H., Swigris, J. J., Wells, A. U., Ancochea, J., Bouros, D., Carvalho, C., Costabel, U., Ebina, M., Hansell, D. M., Johkoh, T., Kim, D. S., King, T. E., Kondoh, Y., Myers, J., Muller, N. L., Nicholson, A. G., Richeldi, L., Selman, M., Dudden, R. F., Griss, B. S., Protzko, S. L., Schilnemann, H. J., 2011. An Official ATS/ERS/JRS/ALAT Statement: Idiopathic Pulmonary Fibrosis: Evidence-based Guidelines for Diagnosis and Management. Am. J. Respir. Crit. Care Med. 183, 788-824. doi:10.1164/rccm.2009-040GL
Raghu, G., Rochwerg, B., Zhang, Y., Garcia, C. A. C., Azuma, A., Behr, J., Brozek, J. L., Collard, H. R., Cunningham, W., Homma, S., Johkoh, T., Martinez, F. J., Myers, J., Protzko, S. L., Richeldi, L., Rind, D., Selman, M., Theodore, A., Wells, A. U., Hoogsteden, H., Schünemann, H. J., 2015. An Official ATS/ERS/JRS/ALAT Clinical Practice Guideline: Treatment of Idiopathic Pulmonary Fibrosis. An Update of the 2011 Clinical Practice Guideline. Am. J. Respir. Crit. Care Med. 192, e3-e19. doi:10.1164/rccm.201506-10635T
Raghu, G., Weycker, D., Edelsberg, J., Bradford, W. Z., Oster, G., 2006. Incidence and Prevalence of Idiopathic Pulmonary Fibrosis. Am. J. Respir. Crit. Care Med. 174, 810-816. doi:10.1164/rccm.200602-163OC
Richeldi, L., du Bois, R. M., Raghu, G., Azuma, A., Brown, K. K., Costabel, U., Cottin, V., Flaherty, K. R., Hansell, D. M., Inoue, Y., Kim, D. S., Kolb, M., Nicholson, A. G., Noble, P. W., Selman, M., Taniguchi, H., Brun, M., Le Maulf, F., Girard, M., Stowasser, S., Schlenker-Herceg, R., Disse, B., Collard, H. R., 2014. Efficacy and Safety of Nintedanib in Idiopathic Pulmonary Fibrosis. N. Engl. J. Med. 370, 2071-2082. doi:10.1056/NEJMoa1402584
Thomeer, M. J., Costabel, U., Rizzato, G., Poletti, V., Demedts, M., 2001. Comparison of registries of interstitial lung diseases in three European countries. Eur. Respir. J. 18, 114s-118s.
Van der Aar E M, Fagard L, Desrivot J, Dupont S, Heckmann B, Blanque R, Gheyle L, Ralic J, Vanhoutte F. Favorable human safety, pharmacokinetics, and pharmacodynamics of the autotoxin inhibitor GLP1690, a potential new treatment in IPF. Poster presented at: American Thoracic Society Conference. 2016 May 13-18; San Francisco, Calif.

1. A method of treating fibrotic diseases, said method comprising the steps of:
a) obtaining three-dimensional image data of a subject's respiratory system, which image data has been acquired during an assessment period, wherein the assessment period comprises a breathing cycle and the image data comprise high-resolution computer tomography (CT) high-resolution images at functional residual capacity (FRC) and high-resolution computer tomography (CT) images at total lung capacity (TLC);
b) calculating a specific three-dimensional structural model of the subject's respiratory system using the three-dimensional image data of step a) to determine one or more outcome parameters as described in WO2014125059;
c) administering a daily dose of between 100 mg to 1000 mg to the subject of a compound according to Formula I (Cpd 1), or a pharmaceutically acceptable salt thereof: 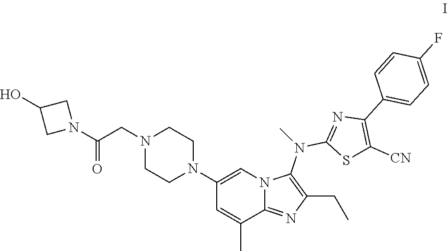 [SMILES: CCc1nc2c(C)cc(N3CCN(CC(=O)N4CC(O)C4)CC3)cn2c1N(C)c1nc(-c2ccc(F)cc2)c(C#N)s1]
d) repeating steps a and b above after administration of the compound;
e) comparing the three-dimensional structural models and/or outcome parameters of step b) and d) with each other;
f) calculating the response to the treatment of said subject from step e);
g) determining whether the dose of the compound according to Formula I should be increased, decreased or maintained at the same level based on the results obtained in step f).

2. The method according to claim 1, wherein the specific three-dimensional structural model of the subject's respiratory system comprises a three-dimensional structural model of the subject's lung lobar structure and a three-dimensional structural model of the subject's airway structure.

3. The method according to claim 1, wherein the one or more outcome parameters comprise the lobar volume, preferably at FRC and TLC; or wherein the one or more outcome parameters comprise the airway volume, preferably at FRC and TLC; or wherein the one or more outcome parameters comprise lobar emphysema; or wherein the one or more outcome parameters comprise lobar blood vessel volume; or wherein the one or more outcome parameters comprise the airway wall thickness; or wherein the one or more outcome parameters comprise the airway resistance, preferably at FRC and TLC; or wherein the one or more outcome parameters comprise the airway volume and/or resistance.

4. The method according to claim 3, wherein the one or more outcome parameters comprise the airway volume and/or resistance.

5. The method according to claim 1, wherein the compound of step c) is administered over a period of at least 1 week, at least 2 weeks, at least 4 weeks, at least 8 weeks or at least 12 weeks.

6. The method according to claim 1, wherein the compound of step c) is administered over a period of at least 12 weeks.

7. The method according to claim 1, wherein step d is performed after 4 weeks, after 8 weeks, or after 12 weeks administration of the compound according to formula I.

8. The method according to claim 1 or 5, wherein the compound, or a pharmaceutically acceptable salt thereof of step c) is administered at a dose of 600 mg qd.

9. The method according to any one of claims 1-7, comprising measuring the forced vital capacity FVC in the subject, wherein the FVC does not decrease compared to after treatment.

10. The method according to any one of claims 1-7, comprising measuring the forced vital capacity FVC in the subject, wherein said FVC increases by at least 1 mL, at least 2 mL, at least 3 mL, at least 4 mL, at least 5 mL, at least 6 mL, at least 7 mL or at least 8 mL over a period of 12 weeks.

11. The method according to any one of claims 1-7, comprising measuring the airway volume wherein said airway volume decrease is no more than 5 mL/L, no more than 4 mL/l, or no more than 3 mL/L after 12 weeks.

12. The method according to any one of claims 1-7, comprising measuring the airway resistance wherein said airway resistance increase is at least 0.05 kPa/s, at least 0.06 kPa/s, at least 0.07 kPa/s, at least 0.08 kPa/s, at least 0.09kPa/s, or at least 1.0 kPa/s after 12 weeks.

13. The method according to claim 1, wherein the compound or a pharmaceutically acceptable salt thereof of step c) is administered as a pharmaceutical composition.

14. The method according to claim 12, wherein the pharmaceutical composition comprises a pharmaceutically acceptable carrier, and an effective amount of the compound according to Formula I.

15. The method according to claim 1, wherein the fibrotic disease is IPF.

16. A method of treating idiopathic pulmonary fibrosis comprising the steps of administering a subject a daily dose of compound according to formula I of 100 mg or more, such as 100 to 1000 mg, 100 to 600 mg, 200 to 1000 mg, or, 200 to 600 mg.

17. A method according to claim 16 wherein the daily dose is a single dose of 600 mg of compound according to formula I.

18. A method of treating idiopathic pulmonary fibrosis comprising the steps of administering a subject a daily dose of 200 mg of compound according to formula I.

19. A method according to any of claims 16 to 18, further comprising measuring forced vital capacity (FVC) in the subject, wherein said FVC does not decrease following treatment.

20. A method according to any of claims 16 to 18, further comprising measuring forced vital capacity (FVC) in the subject, wherein said FVC does not decrease following 12 weeks of treatment.

21. A method according to claim 19 or 20, wherein said FVC increases by at least 1 mL, at least 2 mL, at least 3 mL, at least 4 mL, at least 5 mL, at least 6 mL, at least 7 mL or at least 8 mL over a period of 12 weeks.

22. A method according to any of claims 16 to 21, further comprising measuring the airway volume wherein said airway volume decrease is no more than 5 mL/L, no more than 4 mL/l, or no more than 3 mL/L after 12 weeks.

23. A method according to any of claims 16 to 22, further comprising measuring the airway resistance wherein said airway resistance increase is at least 0.05 kPa/s, at least 0.06 kPa/s, at least 0.07 kPa/s, at least 0.08 kPa/s, at least 0.09kPa/s, or at least 1.0 kPa/s after 12 weeks.

24. A compound according to formula I or a pharmaceutically acceptable salt thereof for use in any of the methods of claims 1 to 23.
